(19) United States
(12) Plant Patent Application Publication
(10) Pub. No.: US 2003/0070195 P1
(43) Pub. Date: Apr. 10, 2003

(54) Floribunda rose plant named 'Meilomit'

(76) Inventor: Alain A. Meilland, Antibes (FR)

Correspondence Address: BURNS, DOANE, SWECKER & MATHIS, L.L.P., P.O. Box 1404, Alexandria, VA, 22313-1404, US

(21) Appl. No.: 10/299,914

(22) Filed: Nov. 20, 2002

Related U.S. Application Data
(63) Continuation of application No. 09/573,163, filed on May 19, 2000, now abandoned.

(51) Int. Cl. A01H 5/00

(52) U.S. Cl. PLT/146

(57) ABSTRACT

A new and distinct variety of Floribunda rose plant is provided which forms in abundance attractive double blossoms that are Marigold Orange suffused with red in coloration. Such blossoms possess a light fragrance. The plant exhibits a bushy growth habit, and forms decorative dense dark green foliage that contrasts nicely with the blossom coloration. The plant also maintains well its attractive appearance even at the end of the blooming season. The new variety is particularly well suited for providing colorful ornamentation in the landscape.

### SUMMARY OF THE INVENTION

The new Rosa hvbrida variety of Floribunda rose plant was created by artificial pollination wherein two parents were crossed which previously had been studied in the hope that they would contribute the desired characteristics. The female parent (i.e., the seed parent) of the new variety was the ‘Meigurami’ variety (U.S. Plant Pat. No. 4625). The male parent (i e., the pollen parent) was the product of the cross of the ‘Allgold’ variety (U.S. Plant Pat. No. 1665) and the ‘Yellow Cushion’ variety (U.S. Plant Pat. No. 2849). The parentage of the new variety can be summarized as follows:

‘MeiguramI’×(‘Allgold’×‘Yellow Cushion’).

The seeds resulting from the above pollination were sown and small plants were obtained which were physically and biologically different from each other. Selective study resulted in the identification of a single plant of the new variety.

It was found that the new variety of Floribunda rose plant of the present invention possesses the following combination of characteristics:

(a) forms in abundance attractive double blossoms that are Marigold Red suffused with red in coloration,

(b) exhibits a bushy growth habit,

(c) forms decorative dense dark green glossy foliage, and

(d) is particularly well suited for growing as ornamentation in parks and gardens.

The blossom coloration contrasts nicely with the dark green foliage. Also, the plant maintains well its attractive appearance even at the end of the blooming season.

The new variety well meets the needs of the horticultural industry and is particularly well suited for growing in the landscape.

The new variety has been found to undergo asexual propagation in France by a number of routes, including budding, grafting, and cuttage. Asexual propagation by the above-mentioned techniques in the South of France has shown that the characteristics of the new variety are stable and are strictly transmissible by such asexual propagation from one generation to another.

The new variety has been named the ‘Meilomit’ variety.

### BRIEF DESCRIPTION OF THE PHOTOGRAPH

The accompanying photograph shows as nearly true as it is reasonably possible to make the same, in a color illustration of this character, typical specimens of the plant parts of the new variety. The rose plants of the new variety were two years of age and were observed during September while budded on Rosa froebelli understock and growing outdoors at Le Cannet des Maures, Var, France. Dimensions in centimeters are indicated at the bottom of the photograph.

FIG. 1—illustrates a specimen of a young shoot;

FIG. 2—illustrates a specimen of a floral bud before the opening of the sepals;

FIG. 3—illustrates a specimen of a floral bud at the opening of the sepals;

FIG. 4—illustrates a specimen of a floral bud at the opening of the petals;

FIG. 5—illustrates a specimen of a flower in the course of opening;

FIG. 6—illustrates a specimen of an open flower—plan view—obverse;

FIG. 7—illustrates a specimen of an open flower—plan view—reverse;

FIG. 8—illustrates a specimen of a fully open flower—plan view—obverse;

FIG. 9—illustrates a specimen of a fully open flower—plan view—reverse;

FIG. 10—illustrates a specimen of a floral receptacle showing the arrangement of the stamens and pistils;

FIG. 11—illustrates a specimen of a floral receptacle showing the arrangement of the pistils (stamens removed);

FIG. 12—illustrates a specimen of a flowering stem;

FIG. 13—illustrates a specimen of a main branch;

FIG. 14—illustrates a specimen of a leaf with three leaflets—plan view—upper surface;

FIG. 15—illustrates a specimen of a leaf with five leaflets—plan view—under surface; and

FIG. 16—illustrates a leaf with seven leaflets—plan view—upper surface.

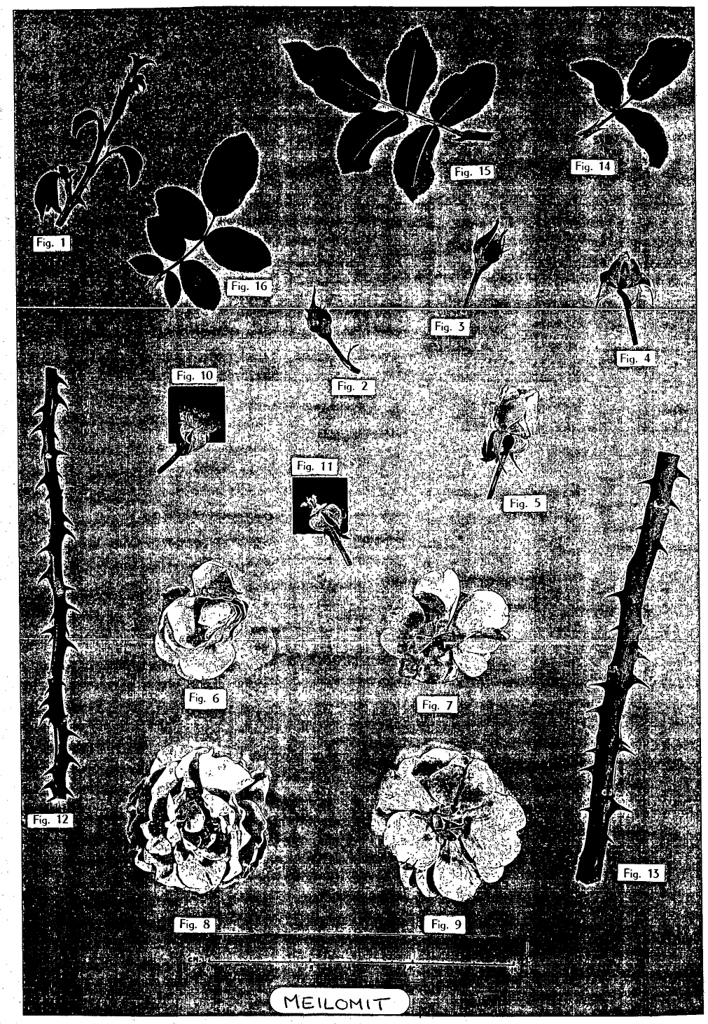

### DETAILED DESCRIPTION

The chart used in the identification of the colors is that of the Royal Horticultural Society (R.H.S. Colour Chart). The description is based on the observation of two year-old plants during October while budded on Rosa froebelli understock and growing outdoors at Le Cannet des Maures, Var, France. The coloration in common terms precedes reference to the chart in some instances.

CLASS: Floribunda.

PLANT: height—approximately 60 to 70 cm. on average at the end of the growing season. habit—bushy.

BRANCHES: color—young stems: Yellow-Green Group 147A and stained with reddish-brown coloration. adult wood: Green Group 138A.

thorns.—size: medium (as illustrated). quantity: numerous (as illustrated). color: reddish on young stems and tan on adult wood. configuration: deeply concave.

LEAVES: stipules—adnate, pectinate, and rather broad.

petioles.—upper surface: glabrous and near Green Group 137A in coloration. under surface: slightly glandular, and near Yellow-Green Group 148 A in coloration.

leaflets.—number: 3,5, and 7. shape: elliptic. serration: regular (as illustrated). texture: rather consistent. general appearance: very dense, dark green, and glossy. color (young foliage):

upper surface: dark green, near Green Group 136 A. under surface: dark green, near Green Group 137 A with some reddish anthocyanin coloration. color (adult foliage): upper surface: dark green, near Green Group 139 A. under surface: dark green, near Green Group 137 A.

### INFLORESCENCE

number of flowers.—usually two to five flowers per stem.

peduncle.—rigid and short.

sepals.—upper surface: tomentose, near Yellow-Green Group 148 C and more or less suffused with reddish anthocyanin coloration. under surface: glabrous, near Yellow-Green Group 146 A and more or less suffused with reddish anthocyanin coloration. Some small extensions are present (as illustrated).

buds.—shape: conical. size: medium. length: approximately 2 cm. on average.

flower.—shape: flat cup-shaped. diameter: commonly approximately 8.5 to 9.5 cm. color (when opening begins): upper surface: Marigold Orange, near Orange Group 28 C and lightly suffused with Red Group 52 D. under surface: Marigold Orange, near Orange Group 28 C and lightly suffused with Red Group 52 D. color (when blooming): upper surface: Marigold Orange, near Orange Group 28 C and lightly suffused with Red Group 52 D. under surface: Marigold Orange, near Orange Group 28 C and lightly suffused with Red Group 52 D. color (at end of opening): upper surface: near Orange Group 29 D at the base and near Red Group 49 D at the tip. under surface: near Orange Group 29 D at the base and near Red Group 55 D at the tip. fragrance: light. lasting quality: long on the plant. petal shape: rounded. stamen number: approximately 95 on average. anthers: near Orange Group 29 C in coloration. filaments: near Yellow Group 8 B in coloration. pistils: approximately 32 on average. stigmas: near Yellow Group 11 A in coloration. styles: near Yellow Group 11 D in coloration. receptacle: near Yellow-Green Group 146 A in coloration, and in longitudinal section in the shape of a pitcher.

### DEVELOPMENT

vegetation.—strong.

blooming.—very abundant. resistance to

diseases.—excellent.

aptitude to.—

bear fruit.—good, forms rounded fruit.

I claim:

1. A new and distinct variety of Floribunda rose plant characterized by the following combination of characteristics:
(a) forms in abundance attractive double blossoms that are Marigold Orange suffused with red in coloration,
(b) exhibits a bushy growth habit,
(c) forms decorative dense dark green glossy foliage, and
(d) is particularly well suited for growing as ornamentation in parks and gardens; substantially as herein shown and described.
